(19) United States
(12) Patent Application Publication
(10) Pub. No.: US 2007/0167899 A1
(43) Pub. Date: Jul. 19, 2007

(54) Compression apparatus and method for stimulating hair growth

(76) Inventor: Randy Allen Strom, Chandler, AZ (US)

Correspondence Address: RANDY A. STROM, 2040 S. ALMA SCHOOL RD STE. 1 #172, CHANDLER, AZ, 85248, US

(21) Appl. No.: 11/334,791

(22) Filed: Jan. 19, 2006

(51) Int. Cl. A61L 15/00

(52) U.S. Cl. 602/75

(57) ABSTRACT

A compression apparatus for reducing hair loss and stimulating hair growth on the scalp of a human subject by producing a shift in the scalp upward and forward by compression from connected stretch type cloth straps that loop around the subjects head. The inner side of the top of the apparatus has coarse pads added to produce friction with the scalp and hair. The top strap is connected to the top of the two side straps with two rings. The bottom of these two side straps is connected to a chinstrap with a hook-and-loop type fastener. In addition, there are optional side supports that can be added to increase the amount of scalp being shifted.

### BACKGROUND OF THE INVENTION—PRIOR ART

Finding the cure for baldness and thinning hair on both men and women has long been a quest for many inventors. There has been many inventions to remedy such a problem, but very few with only some to limited results. These inventions range from massaging devices to topical applications and from sophisticated to the obscured. The biggest folly for these inventions is that there has never been a definite answer to the question of why baldness or hair thinning occurs. There is a wide range of possibilities that may be the reason for baldness or thinning hair, such as; genes, illness, medications, internal toxins, DHT, aging and stress.

The most popular and widely used inventions used in the market place, are the topical applications that contain minoxidil or minoxidil derivatives. U.S. Pat. No. 6,596,266 to Catalfo (2003) and U.S. Pat. No. 5,183,817 to Bazzano (1993) are two examples of such inventions. However these solutions provide only limited to no results, are expensive, carry the risk of side effects to the user, and have to be applied regularly for as long as the user wants to maintain the perceived results. An oral administration, such as U.S. Pat. No. 6,355,649 to Gormley (2002) also carries many of the same follies as the topical applications. It provides only limited to no results, is very expensive, is for men only, carries the risk of many side effects to the user, and have to be taken regularly for as long as the user wants to maintain the perceived results. U.S. Pat. No. 4,969,903 to Valle (1990) uses a technique of hair implanting by plugging synthetic hair or natural hair strands into the scalp where balding or thinning has occurred. This is merely a way of masking baldness and not a cure. It is very expensive, time consuming, can be painful, and may leave scaring.

There are other inventions that may be less known, but strive for the same results. U.S. Pat. No. 6,497,719 to Pearl (2002) uses a laser to combat baldness with limited to no results. This too is expensive, time consuming, and must be used regularly. U.S. Pat. No. 6,834,206 to Pitzen (2003) uses an electrical therapeutic signal, but must be manipulated and administered by the operator of the apparatus.

Another patent, U.S. Pat. No. 5,031,609 to Fye (1991), is a similar looking invention, but serves a different purpose. It is a compression bandage for post rhytidectomy plastic surgery or after a facelift to hold the face stationary. All the heretofore patents have one or more of the following disadvantages:
(a) They are for male pattern baldness, or androgenetic alopecia, and not for the problem of a receding hairline.
(b) They are expensive.
(c) They must be administered or taken daily.
(d) May have numerous, and sometimes harsh side effects.
(e) The results are questionable.
(f) The procedure or application can be uncomfortable or even painful.
(g) The procedure or application contains chemicals.

### BACKGROUND OF INVENTION—OBJECTS AND ADVANTAGES

There are several objects and advantages of the present patent, which are:
(a) to provide a natural way to stimulate hair growth, using no chemicals or medical procedures;
(b) to provide a solution that is relatively inexpensive and uses only one apparatus, which can be used continuously, until the materials wear out or it breaks;
(c) to provide a solution that has no unwanted chemical side effects;
(d) to provide a solution that works on the whole scalp, including a receding hair line;
(e) to provide a solution for both male and female;
(f) to provide a solution that is not time consuming and requires only one person to place it on the head;
(g) to provide a catalyst to other methods of fighting hair loss;
(h) to provide a solution that is non-painful;
(i) to provide a solution that doesn't require daily or routine use.

Further objects and advantages are to provide an easy to use way to stimulate hair growth that is also comfortable to the user.

### SUMMARY

In accordance with the present invention a compression apparatus for reducing hair loss and stimulating hair growth on the head of a human subject comprising of a stretch-type cloth that works by shifting the scalp upward, toward the top of the head, and forward toward the forehead, to un-kink the blood vessels to create an improved blood flow to the scalp and hair.

### DRAWINGS—FIGURES

FIG. 1 shows the compression apparatus.

FIG. 2 shows the compression apparatus with two side supports added.

FIG. 3 shows how the compression apparatus is worn on the human head.

FIG. 4 shows how the compression apparatus is worn on the human head with two side supports added.

### DRAWINGS—REFERENCE NUMERALS

1 top stretch-type cloth

2 ring

3 coarse pad

4 hook-and-loop fastener

5 side stretch-type cloth

6 chin strap

7 side support

8 side coarse pad

9 side support hook-and-loop fastener

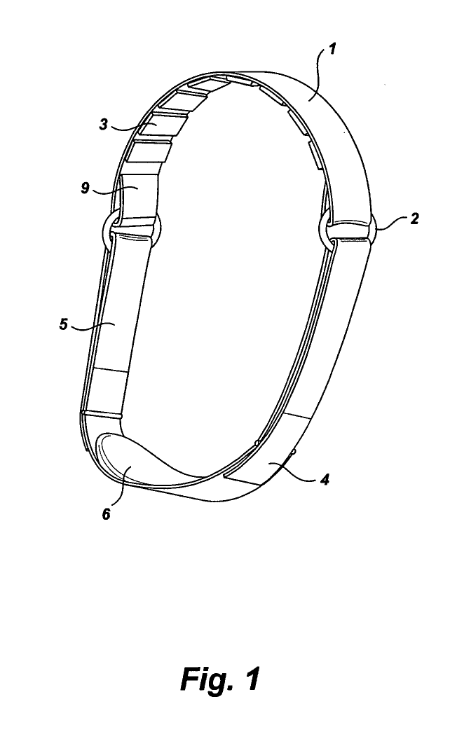

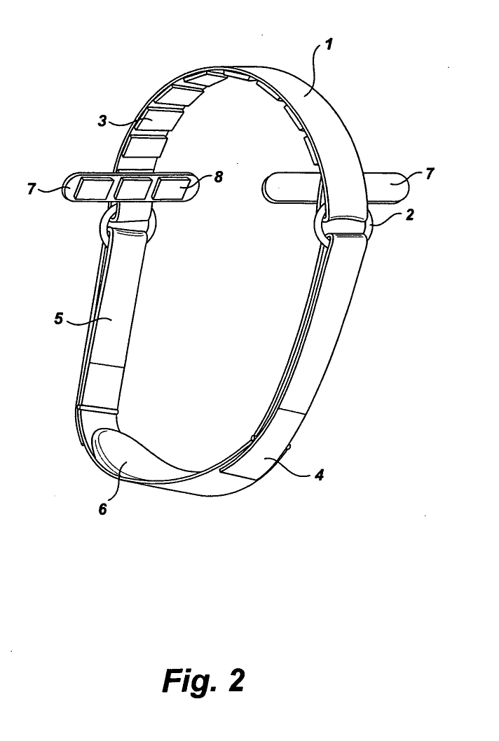

### DETAILED DESCRIPTION—FIGS. 1 AND 2—PREFERRED EMBODIMENT

A preferred embodiment of the compression apparatus of the present invention is illustrated in FIG. 1 (front-left view) and FIG. 2 (front-left view). The apparatus has a stretch-type cloth that fits over the top of the head 1 that is approximately 15 mm long and 2.5 mm wide that connects to two plastic rings 2 that have an approximate radius of 5 mm. There are small coarse pads 3 that are attached to the inner side of 1 that are roughly 2.5 mm by 2.5 mm. The two rings are then attached to two stretch-type cloth side straps 5 that are approximately 18 mm long by 2.5 mm wide and have a hook-and-loop fastener 4 attached to their ends. The two hook-and-loop fastener ends are then attached to a chinstrap 6 that is made of cloth and synthetic fur and is approximately 12.5 mm long by 7.5 mm wide. There are two removable side supports 7 that are made of plastic and are approximately 7.5 mm long by 1.5 mm wide with coarse pads 8 attached to the inner side. These two removable side supports are each attached to the inner side of the top stretch-type cloth by a hook-and-loop fastener 9.

Operation—FIGS. 1, 2, 3, 4

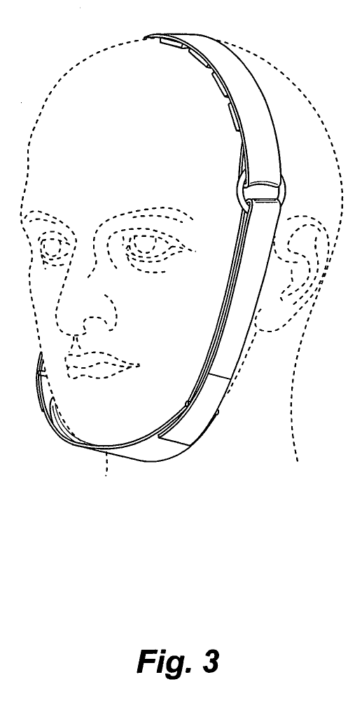

To place the compression apparatus on, the user places the top stretch-type cloth 1 over the top of the head, in the direction of ear to ear. You then hold this top stretch-type cloth down on the head with one hand, and stretch the bottom chinstrap 6 down and around the chin with the other hand. The apparatus should be snug, from the top of the head and down around the chin (FIG. 3). To remove the apparatus, the user simply pulls it off the head with there hand. If the two side supports are added, the compression apparatus is placed on and taken off in the same manner as without the side supports added.

The compression apparatus should not be worn to tightly or to loosely. Once the apparatus is on, you test to make sure it is on correctly by gently lifting up on the top portion of the forehead, around the front hair line, with your fingers, to make sure there is give in the skin. One should be able to move the forehead skin and scalp in a circular motion with ease. If the apparatus does not fit snug, then adjust its size with one, or both, of the hook-and-loop fasteners 4. The compression apparatus can be worn with the head in any position, and while doing almost any activity. The amount of force and the direction of the shift will vary depending on how tight the compression apparatus is on, and the angle the user has it on the head.

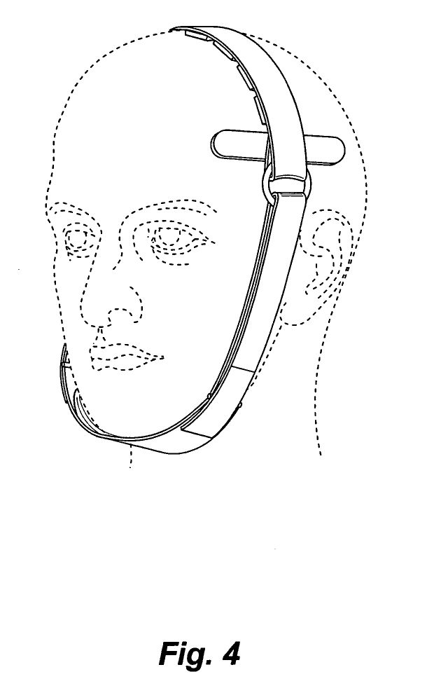

FIGS. 2, 4—Alternative Embodiments

Alternative embodiments that can be added to the compression apparatus are shown in FIG. 3 and 4. These embodiments are the side supports 7 that are added to add more support to the shift in the scalp. These side supports increase the amount of scalp being shifted and held. They are attached to the inner side of the top stretch-type cloth of the apparatus by hook-and-loop fastener 9. Advantages

From the description above, a number of advantages of my compression apparatus become evident:
(a) This is a natural way to stimulate the scalp to promote hair growth and it does not contain any chemicals or require any medical procedures to be done.
(b) The cost of the compression apparatus is relatively inexpensive when compared to other methods, and one apparatus can be used continuously until the materials that make up the apparatus wear out.
(c) There are no unwanted chemical side effects, because the compression apparatus contains no such chemicals.
(d) The compression apparatus provides a solution that works on the whole scalp, including a receding hairline.
(e) The compression apparatus provides a solution for both male and female users.
(f) This compression apparatus is easy to use, requires only one person to place it on the head, and is not time consuming.
(g) The compression apparatus can also be used with other methods of hair loss prevention as a catalyst.
   Conclusion, Ramifications, and Scope

Accordingly, the reader will see that the compression apparatus of this invention can be used to stimulate hair growth and reduce hair loss by decreasing the blood flow restriction to the scalp and hair. It is simply worn on the head and is adjustable to fit different head sizes. In addition, this compression apparatus can be worn while performing most activities. Furthermore, the compression apparatus has the additional advantages in that
it provides a natural way to stimulate hair growth, without using chemicals or medical procedures;
it provides a solution that is relatively inexpensive to stimulate hair growth;
it provides a solution that has no unwanted chemical side effects;
it provides a solution that works on the whole scalp, including a receding hair line;
it provides a solution for both male and female;
it provides a solution that is easy to use, is not time consuming, and requires only one person to place it on the head.

Although the description above contains many specifications, these should not be construed as limiting the scope of the invention but as merely providing illustrations of some of the presently preferred embodiments of this invention. For example, the compression apparatus can have other shapes of the parts used, such as square, oval, circle, etc.; made of different materials, such as, elastic, terry cloth, leather, rubber, etc.; made with more or less tension or support.

Thus the scope of the invention should be determined by the appended claims and their legal equivalents, rather then by the examples given.

I claim:

1. A compression apparatus for reducing hair loss and stimulating hair growth on the head of a human subject comprising of at least one stretch-type cloth that wraps around the head, from the top of the said head, and down both sides of the face, and around the chin, which makes a complete loop, as a means for a force by compression on the said head, which produces a shift of the scalp and hair upwards, from the crown of the head to the top of the head, and forward towards the forehead.

2. The apparatus of claim 1 wherein at least one coarse pad is connected to the inner side of the said stretch-type cloth to produce friction with the said scalp and hair.

3. The apparatus of claim 1 wherein at least two rings are connecting at least three of the said stretch-type cloth to help contour to the said head.

4. The apparatus of claim 1 wherein at least one hook-and-loop type fastener is attached to the said stretch-type cloth to adjust the said loop to fit a different size said head.

5. The apparatus of claim 1 wherein a chin pad is added for comfort to the said human subject.

6. The apparatus of claim 1 wherein at least one removable side support is added as a means of shifting more of the said scalp.

7. A compression apparatus for reducing hair loss and stimulating hair growth on the scalp of a human subject comprising of at least one stretch-type cloth that loops around the head to produce a force from compression on the said head, as a means of moving the scalp and hair upward, toward the top of the said head, and forward towards the forehead, to reduce blood flow restriction.

8. The apparatus of claim 7 wherein at least one coarse type pad is connected to the inner side of the said stretch-type cloth, that is in direct contact with the said hair and scalp to produce friction.

9. The apparatus of claim 7 wherein at least two rings are connecting at least three of the said stretch-type cloth to contour to the said head.

10. The apparatus of claim 7 wherein a chin pad is added for comfort to the said human subject.

11. The apparatus of claim 7 wherein at least two removable side supports are added as a means of shifting the said scalp.

12. A method for reducing hair loss and stimulating hair growth on the head of a human, by means of a force on the head to reduce blood flow restriction comprising, of a compression apparatus that shifts the scalp and hair on the head upward, from the sides of the head, from the crown of the head toward the top of the head, and forward toward the forehead.

13. The apparatus of claim 12 wherein at least one coarse type pad is connected to the inner side of the said stretch-type cloth to produce friction with the said scalp and hair.

14. The apparatus of claim 12 wherein at least two rings are connecting at least three of the said stretch-type cloth to contour to the said head.

15. The apparatus of claim 12 wherein at least one hook-and-loop type fastener is attached to the said stretch-type cloth to adjust the said loop to fit a different size said head.

16. The apparatus of claim 12 wherein a chin pad is added for comfort.

17. The apparatus of claim 12 wherein at least one removable side support is added as a means of shifting more of the said scalp and hair.
